(19) United States
(12) Patent Application Publication
(10) Pub. No.: US 2016/0331578 A1
(43) Pub. Date: Nov. 17, 2016

(54) SEMINAL AND URINARY FLUID EMISSION ARRESTING DEVICES, SYSTEMS, AND METHODS OF USING THE SAME

(71) Applicant: Momo Sumina, Las Vegas, NV (US)

(72) Inventor: Momo Sumina, Las Vegas, NV (US)

(21) Appl. No.: 14/712,621

(22) Filed: May 14, 2015

(51) Int. Cl. A61F 6/04

(52) U.S. Cl. CPC A61F 6/04; A61F 2006/043; A61F 2006/047

(57) ABSTRACT

A fluid emission arresting device is provided. In some embodiments, the fluid emission arresting device may comprise: a barrier layer made of a flexible material which may comprise a generally planar exterior surface and a generally planar interior surface. An adhesive layer may be applied to the interior surface of the barrier layer and may be configured to attach to the glans of the penis to position the barrier layer over the urethra opening to block fluid from exiting the urethra opening. In further embodiments, once the device is attached to the penis over the urethra opening, the barrier layer may prevent fluid from exiting the urethra so that the urethra may function as a fluid reservoir.

### CROSS REFERENCE TO RELATED APPLICATIONS

This application claims priority to and the benefit of the filing date of U.S. Provisional Application No. 62/038,832, filed on Aug. 18, 2014, entitled “Urethra Shield—Alternative to the Male Condom”, which is hereby incorporated by reference in its entirety.

### FIELD OF THE INVENTION

This patent specification relates to the field of contraceptive and incontinence devices. More specifically, this patent specification relates to contraceptive and incontinence devices configured to arrest emissions from the penis during use.

### BACKGROUND

The conventional contraceptive condom has many drawbacks. Since it must be applied while the penis is erect, the sexual experience typically must be halted in order to locate and then apply the condom. These condoms also must be applied from a certain side which can result in confusion during the application as to whether the condom is being applied backwards. This confusion is especially prevalent in low light environments.

Pregnancy and/or disease transmission commonly results because of the inconvenience of locating and then application of the condom before erection/passion wanes and dies. This is due to the period of time between sexual passion arousal and when passion withers. The human species prefers spontaneous love making, which is why most unprepared users simply decide to go without a condom and take a chance. However, a large number of users simply dislike the feel of condoms and decide to “take a chance” anyway. This type of behavior is the primary cause of typical use failure (as opposed to method or perfect use failure).

Standard condoms will fit almost any penis, but with varying degrees of comfort or risk of slippage. Many condom manufacturers offer “snug” or “magnum” sizes. Some manufacturers also offer custom sized-to-fit condoms, with claims that they are more reliable and offer improved sensation/comfort. Some studies have associated larger penises and smaller condoms with increased breakage and decreased slippage rates (and vice versa), but other studies have been inconclusive.) These condoms also tend to slip off the penis when firmness is lost. To this end, the traditional condom is unable to be used for incontinence purposes since it is unable to remain on a flaccid penis.

Since conventional condoms are typically made from types of rubber or membranes, they have a noticeable smell and taste which is not only unpleasant, but can be a health hazard for people with allergies to these materials. Furthermore, materials which are used to make conventional condoms undergo degradation due to the passage of time, improper storage, and/or exposure to incompatible lubricants. This results in the need to constantly check the expiration date of the condom which is also difficult in low light environments.

Therefore, a need exists for novel devices which may be used to prevent conception and the transmission of sexual diseases. There is a further need for novel contraception devices which may also be used for incontinence purposes. A further need exists for novel contraception devices which are not able to be detected by male or female sexual organs during the sexual experience. Finally, there exists a novel contraceptive and incontinence devices which are able to be applied and remain in place on a flaccid as well as an erect penis.

### BRIEF SUMMARY OF THE INVENTION

A fluid emission arresting device is provided. In some embodiments, the fluid emission arresting device may comprise: a barrier layer made of a flexible material which may comprise a generally planar exterior surface and a generally planar interior surface. An adhesive layer may be applied to the interior surface of the barrier layer and may be configured to attach to the glans of the penis to position the barrier layer over the urethra opening to block fluid from exiting the urethra opening.

In further embodiments, once the device is attached to the penis over the urethra opening, the barrier layer may prevent fluid from exiting the urethra so that the urethra may function as a fluid reservoir.

In further embodiments, a fluid emission arresting device may further comprise a dispensing sheet configured to be temporarily secured to the adhesive layer.

In still further embodiments, a fluid emission arresting device may further comprise a protective film configured to be temporarily secured over the exterior surface of the barrier layer to a dispensing sheet.

According to one aspect consistent with the principles of the invention, a method for arresting fluid emission from a penis is provided. The method may include the steps of: removing an adhesive layer from a dispensing sheet of a fluid emission arresting device comprising an adhesive layer, a barrier layer, and a dispensing sheet; applying the adhesive layer to penis to cover urethra opening; and blocking fluid from exiting the urethra with a barrier layer of a fluid emission arresting device affixed over the urethra opening by the adhesive layer.

### BRIEF DESCRIPTION OF THE DRAWINGS

Some embodiments of the present invention are illustrated as an example and are not limited by the figures of the accompanying drawings, in which like references may indicate similar elements and in which:

FIG. 1—FIG. 1 depicts a top perspective view of an example of a fluid emission arresting device according to various embodiments described herein.

FIG. 2—FIG. 2 illustrates a top plan view of an example of a fluid emission arresting device according to various embodiments described herein.

FIG. 3—FIG. 3 shows a top plan view of an alternative example of a fluid emission arresting device according to various embodiments described herein.

FIG. 4—FIG. 4 depicts a top plan view of an alternative example of a fluid emission arresting device according to various embodiments described herein.

FIG. 5—FIG. 5 illustrates a top plan view of an alternative example of a fluid emission arresting device according to various embodiments described herein.

FIG. 6—FIG. 6 shows a perspective view of an example of a fluid emission arresting device secured to a dispensing sheet according to various embodiments described herein.

FIG. 7—FIG. 7 depicts a perspective view of an example of a fluid emission arresting device partially removed from a dispensing sheet according to various embodiments described herein.

FIG. 8—FIG. 8 illustrates an elevation view of an example of a fluid emission arresting system according to various embodiments described herein.

FIG. 9—FIG. 9 shows a sectional, through line A-A shown in FIG. 8, elevation view of an example of a fluid emission arresting system according to various embodiments described herein.

FIG. 10—FIG. 10 depicts a sectional, through line A-A shown in FIG. 8, elevation view of an example of a fluid emission arresting system blocking seminal fluid from exiting the urethra according to various embodiments described herein.

FIG. 11—FIG. 11 illustrates a sectional, through line A-A shown in FIG. 8, elevation view of an example of a fluid emission arresting system blocking urine fluid from exiting the urethra according to various embodiments described herein.

FIG. 12—FIG. 12 shows a plan view of an example of a fluid emission arresting device and a protective film secured to a dispensing sheet according to various embodiments described herein.

FIG. 13—FIG. 13 depicts a plan view of an example of a plurality of fluid emission arresting devices secured to a dispensing sheet according to various alternative embodiments described herein.

FIG. 14—FIG. 14 illustrates a block diagram of an example of a method of arresting seminal fluid emission with a fluid emission arresting system according to various embodiments described herein.

FIG. 15—FIG. 15 shows a block diagram of an example of a method of arresting urine fluid emission with a fluid emission arresting system according to various embodiments described herein.

FIG. 16—FIG. 16 depicts a top plan view of an alternative example of a fluid emission arresting device according to various embodiments described herein.

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

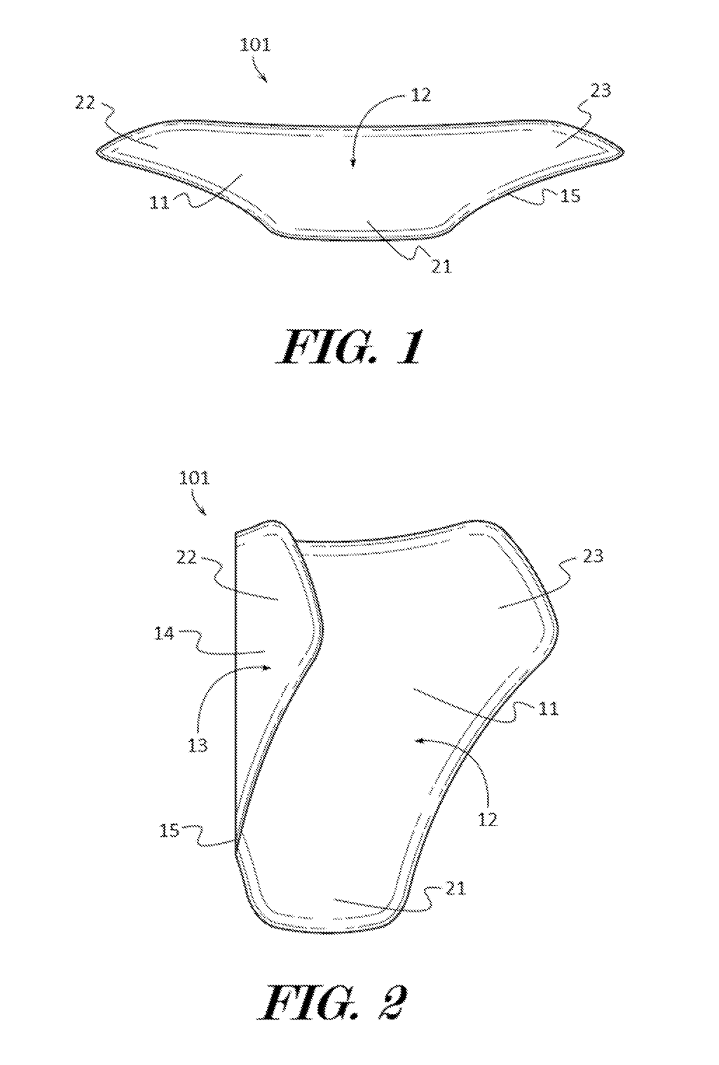

For purposes of description herein, the terms “upper”, “lower”, “left”, “rear”, “right”, “front”, “vertical”, “horizontal”, and derivatives thereof shall relate to the invention as oriented in FIG. 1. However, one will understand that the invention may assume various alternative orientations and step sequences, except where expressly specified to the contrary. Therefore, the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

New contraceptive and incontinence devices, systems, and methods are discussed herein. In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention, and is not intended to limit the invention to the specific embodiments illustrated by the figures or description below.

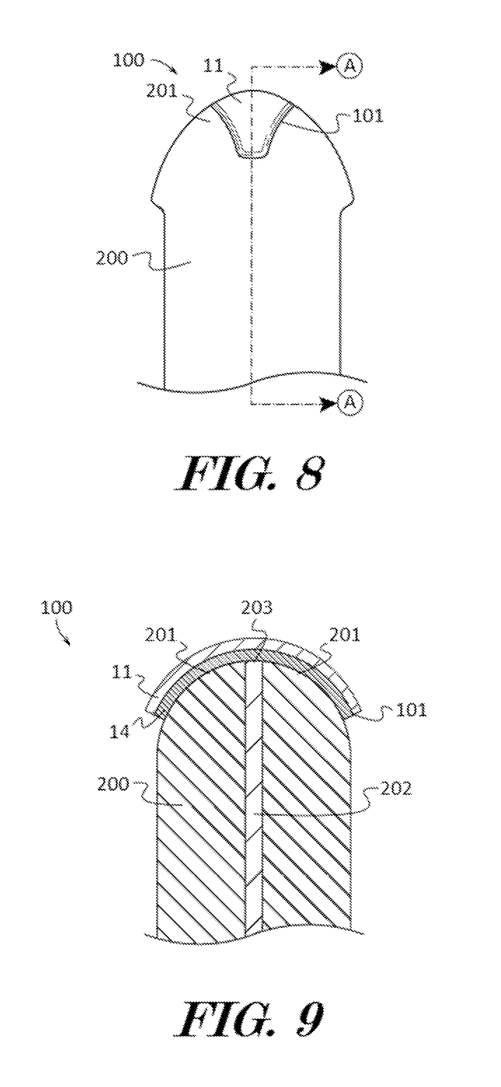

The present invention will now be described by example and through referencing the appended figures representing preferred and alternative embodiments. FIGS. 1 and 2 illustrates an example of an example of a fluid emission arresting device (“the device”) 101 according to various embodiments. In this example, the device 101 comprises a barrier layer 11 with an exterior surface 12 and an interior surface 13. An adhesive layer 14 may be applied to the interior surface 13. The barrier layer 11 may comprise a thin film of material which is generally impermeable to fluids, bacteria, viruses, and other sexually transmitted pathogens. The adhesive layer 14 may be applied to the entire or portions of the interior surface 13 and may be of such as type that it is able to be removably secured or affixed to human skin. Additionally, the adhesive layer 14 comprises an adhesive that is not dissolved by body fluids, such as blood, urine, seminal fluid, and the like. The device 101 may be secured to the penis 200 (FIGS. 8 and 9) so that the adhesive layer 14, and therefore the interior surface 13, may be affixed to the glans 201 (FIGS. 8 and 9) over the urethra 202 (FIGS. 8 and 9). Once the device 101 is secured to the glans 201 over the urethra 202, emissions of pathogens and bodily fluids, such as seminal fluid and urine, may be prevented from exiting the urethra 202 by the barrier layer 11. The barrier layer 11 is also configured to prevent bodily fluids and pathogens from entering the urethra 202.

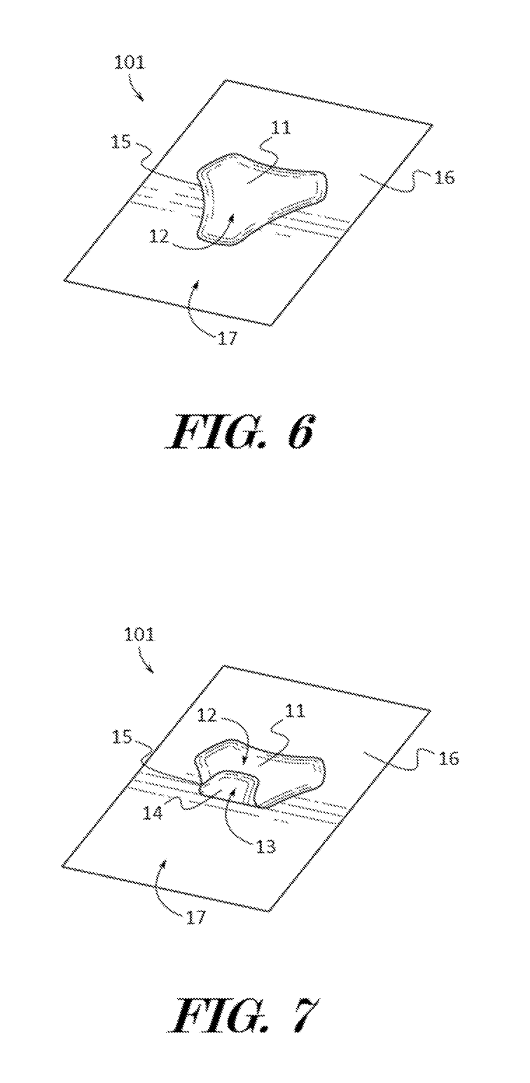

The barrier layer 11 may comprise a generally flexible film of material that is impermeable to body fluids, such as urine and seminal fluid, and to pathogens, such as viruses, bacteria, prions, fungi, viroids, parasites, and the like. The barrier layer 11 may also comprise a generally planar shape which may be made with a flexible material allowing the barrier layer 11 to conform to curved surfaces such as are found on the penis proximate to the opening of the urethra 202 (FIG. 9). In some embodiments, the barrier layer 11 may be made from or comprise a film of polyurethane, polyethylene, polyester, or any other suitable plastic film which is able to flex and is impermeable to body fluids and pathogens. In other embodiments, the barrier layer 11 may be made from or comprise a film of latex rubber, other types of rubber, silicone, plant based plastics, or any other suitable material which may be made into a film and which is able to flex and is impermeable to body fluids and pathogens. In some embodiments, the barrier layer 11 may comprise a pull tab or the like which may be grasped and used to remove the fluid emission arresting device 101 from a dispensing sheet 16 (FIGS. 6 and 7) and/or from the penis 200 (FIGS. 8 and 9).

The adhesive layer 14 may comprise a generally flexible film of adhesive impermeable to body fluids so that it does not dissolve in bodily fluid such as, such as urine, blood, and seminal fluid allowing it to remain affixed to skin in the presence of these fluids. The adhesive layer 14 may be made from or comprise a medical grade adhesive safe for contacting human skin. In some embodiments, the adhesive layer 14 may comprise an acrylic water-based medical grade adhesive, a hot melt medical grade adhesive, a solvent based pressure sensitive adhesive (PSA), or any other suitable type of adhesive. In further embodiments, the adhesive layer 14 may comprise or be made of medical grade adhesive materials that have been approved for direct contact with human skin and are suitable to be applied and removed without damaging the skin.

The adhesive layer 14 may be applied to portions of the interior surface 13 of the barrier layer by being sprayed, squirted, extruded, melted, or any other method of applying adhesive. In some embodiments, the adhesive layer 14 may be applied to portions of the interior surface 13 such as around the perimeter 15 of the interior surface 13. In other embodiments, the adhesive layer 14 may be applied to portions of the interior surface 13 such over the entire interior surface 13.

As shown in FIGS. 1, 2, 6, 7, 10, and 11, in some embodiments, the device 101 may comprise a substantially triangular shape which may be formed by a first lobe 21, a second lobe 22, and a third lobe 23. A triangular shaped device 101 may be configured to attach to portions of the glans 201 (FIGS. 8 and 9) proximate to the urethra opening 203 (FIG. 9) comprising a T-shape. By applying the device 101 to the glans 201 so that the adhesive layer 14 contacts and adheres to the portions of the glans 201 proximate to the urethra opening 203, the lobes 21, 22, 23 may be secured to areas of the glans 201 proximate and surrounding the urethra opening 203 thereby securing the interior surface 13 of the barrier layer 11 over the urethra opening 203 so that any fluid in the urethra 202 may be prevented from exiting the urethra opening 203.

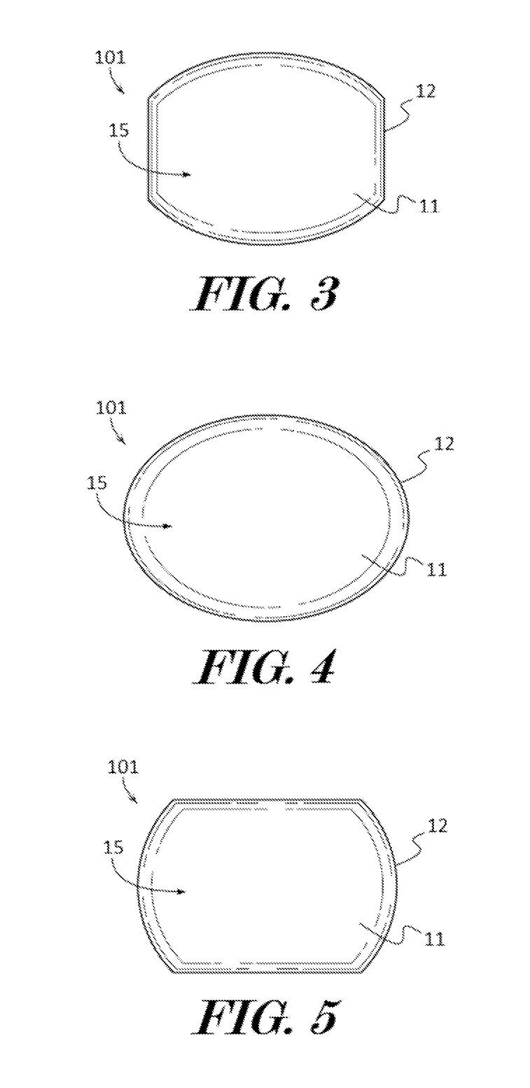

In other embodiments, the device 101 may comprise a general oval shapes (FIG. 3), an ellipse shape (FIG. 4), a stadium shape (FIG. 5), a cuboid shape, a circular shape, a novelty shape such as a five pointed star shape, a silhouette of lips shape, a banana shape, or any other geometric or non-geometric shape, including combinations of shapes. In further embodiments, the device 101 may comprise any shape which is able to conform and cover the portions of the glans 201 proximate to the urethra opening 203, so that the adhesive layer 14 may be secured over the urethra opening 203 thereby preventing fluid in the urethra 202 from exiting the urethra opening 203 and/or any fluid from entering the urethra opening 203. It is not intended herein to mention all the possible alternatives, equivalent forms or ramifications of the invention. It is understood that the terms and proposed shapes used herein are merely descriptive, rather than limiting, and that various changes may be made without departing from the spirit or scope of the invention.

Referring to FIG. 6 and FIG. 7, in some embodiments, the fluid emission arresting device 101 may further comprise a dispensing sheet 16 to which the adhesive layer 14 may be temporarily secured to. A dispensing sheet 16 may provide a protective surface 17 to which the adhesive layer 14 may be temporarily secured to until the device 101 is to be secured to a penis 200. While the adhesive layer 14 is temporarily secured to a dispensing sheet 16 (FIG. 6), dirt and other contaminants may be prevented from contaminating the adhesive layer 14. Additionally, while the adhesive layer 14 is temporarily secured to a dispensing sheet 16, the device 101 may be conveniently transported and transferred by an individual.

In some embodiments, the dispensing sheet 16 may be made from a release liner such as a paper or plastic-based film sheet, to which the adhesive layer 14 may be applied, such as during the manufacturing process, which may be used to prevent the adhesive layer 14 from unwanted adhering to contaminants and other objects. The dispensing sheet 16 may be coated on one or both sides with a release agent such as crosslinkable silicone, other coatings, and materials that have a low surface energy, which provides a release effect against any adhesive which may be used to form the adhesive layer 14. When a user is ready to utilize the device 101, the adhesive layer 14 may be peeled off or otherwise removed from the dispensing sheet 16 as shown in FIG. 7.

Turning now to FIG. 8 and FIG. 9, an elevation view of an example of a fluid emission arresting system 100 and a sectional, through line 9-9 shown in FIG. 8, elevation view of an example of a fluid emission arresting system 100 according to various embodiments described herein are shown, respectively. While the device 101 may be configured with a generally planar shape, the flexible material of the barrier layer 11 and the adhesive layer 14 allow the device 101 to bend to conform to the curve of the glans 201 of the penis 200. The device 101 may be secured to the penis 200 by contacting the adhesive layer 14 to the portions of the glans 201 proximate to the urethra opening 203, so that the adhesive layer 14 may secure the barrier layer 11 over the urethra opening 203. By contacting the adhesive layer 14 to the portions of the glans 201 which encircle the urethra opening 203, fluids are prevented from entering the urethra 202 through the urethra opening 203. Similarly, by contacting the adhesive layer 14 to the portions of the glans 201 which encircle the urethra opening 203, any fluid in the urethra 202 may be prevented from exiting the urethra opening 203. In this manner, bodily fluids in the urethra 202, such as seminal fluid and urine, may be prevented from exiting the urethra opening 203 allowing the urethra 202 to function as a bodily fluid reservoir. Bodily fluids in the reservoir formed by the urethra 202 may therefore be prevented from exiting the urethra 202 until the device 101 is removed from the penis 200.

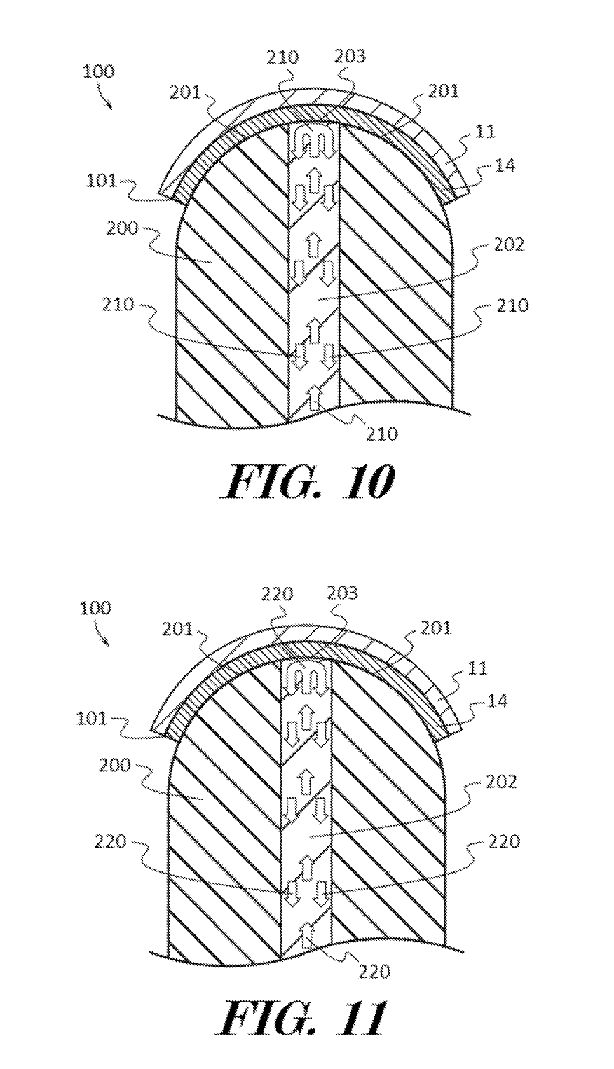

FIG. 10 depicts a sectional, through line A-A shown in FIG. 8, elevation view of an example of a fluid emission arresting system 100 blocking seminal fluid 210 from exiting the urethra 202 according to various embodiments described herein. The fluid emission arresting system 100 may comprise a fluid emission arresting device 101 which may be configured to arrest or prevent bodily fluids, such as seminal fluid 210, from exiting the urethra opening 203 by placing the adhesive layer 14 onto the glans 201 of the penis 200 so that the adhesive layer 14 completely covers the urethra opening 203. The flexible nature of the barrier layer 11 and the adhesive layer 14 allows the device 101 to generally conform to the curved or rounded shape of the glans 201 of a flaccid or erect penis 200. Once the adhesive layer 14 is placed in contact with the area of the glans 201 that surrounds the urethra opening 203, the adhesive layer 14 may maintain the barrier layer 11 over the urethra opening 203. Since the barrier layer is impermeable to bodily fluids, such as seminal fluid 210, any seminal fluid 210 that enters the urethra 202 may then be prevented from exiting the urethra opening 203 while the device 101 is attached to the penis 200 by the adhesive layer 14. In this manner, while the device 101 may not comprise a reservoir, depression, pocket, or the like for containing seminal fluid 210 itself, once the device 101 is attached over the urethra opening 203, the urethra 202 may be configured to act as a reservoir for seminal fluid 210 until the device 101 is removed from covering the urethra opening 203. In some embodiments, the device 101 may be removed by peeling the adhesive layer 14 off of the glans 201, thereby allowing seminal fluid 210 to exit the urethra 202 through the urethra opening 203. In further embodiments, the device 101 may comprise a pull tab which may be grasped and used to pull or peel the adhesive layer 14 off of the glans 201, thereby allowing seminal fluid 210 to exit the urethra 202 through the urethra opening 203. In other embodiments, the glans 201 of the penis 200, may be squeezed or rolled between the user's fingers so that the seminal fluid 210 may be pressurized to force the separation of the adhesive layer 14 and the glans 201, thereby allowing seminal fluid 210 to exit the urethra 202 through the urethra opening 203.

FIG. 11 illustrates a sectional, through line A-A shown in FIG. 8, elevation view of an example of a fluid emission arresting system 100 blocking urine fluid 220 from exiting the urethra 202 according to various embodiments described herein. The fluid emission arresting system 100 may comprise a fluid emission arresting device 101 which may be configured to arrest or prevent bodily fluids, such as urine fluid 220, from exiting the urethra opening 203 by placing the adhesive layer 14 onto the glans 201 of the penis 200 so that the adhesive layer 14 completely covers the urethra opening 203. The flexible nature of the barrier layer 11 and the adhesive layer 14 allows the device 101 to generally conform to the curved or rounded shape of the glans 201 of a flaccid or erect penis 200. Once the adhesive layer 14 is placed in contact with the area of the glans 201 that surrounds the urethra opening 203, the adhesive layer 14 may maintain the barrier layer 11 over the urethra opening 203. Since the barrier layer is impermeable to bodily fluids, such as urine fluid 220, any urine fluid 220 that enters the urethra 202 may then be prevented from exiting the urethra opening 203 while the device 101 is attached to the penis 200 by the adhesive layer 14. In this manner, while the device 101 may not comprise a reservoir, depression, pocket, or the like for containing urine fluid 220 itself, once the device 101 is attached over the urethra opening 203, the urethra 202 may be configured to act as a reservoir for urine fluid 220 until the device 101 is removed from covering the urethra opening 203. In some embodiments, the device 101 may be removed by peeling the adhesive layer 14 off of the glans 201, thereby allowing urine fluid 220 to exit the urethra 202 through the urethra opening 203. In further embodiments, the device 101 may comprise a pull tab which may be grasped and used to pull or peel the adhesive layer 14 off of the glans 201, thereby allowing urine fluid 220 to exit the urethra 202 through the urethra opening 203. In other embodiments, the glans 201 of the penis 200, may be squeezed or rolled between the user's fingers so that the urine fluid 220 may be pressurized to force the separation of the adhesive layer 14 and the glans 201, thereby allowing urine fluid 220 to exit the urethra 202 through the urethra opening 203.

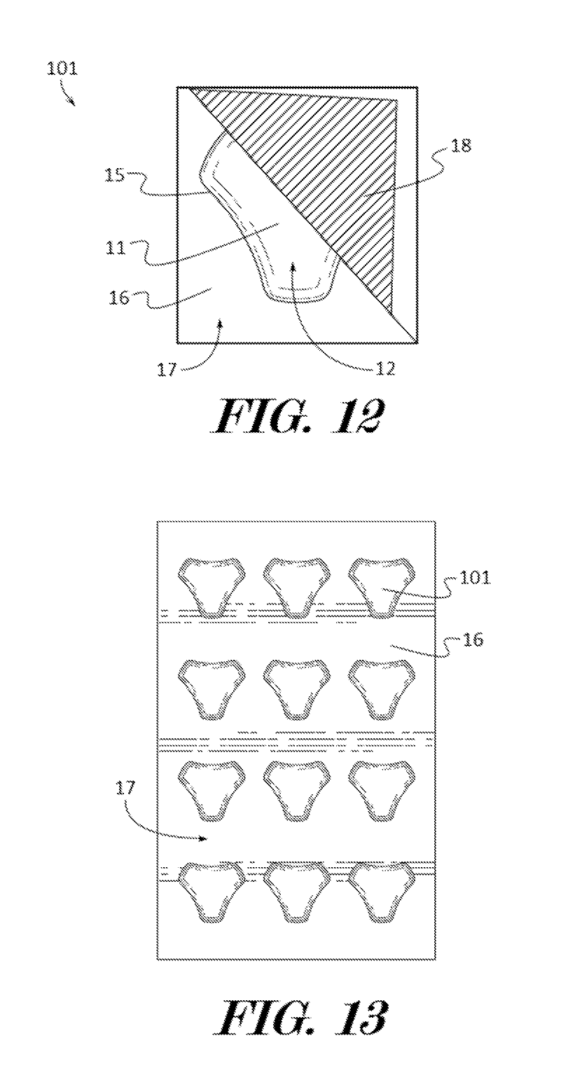

FIG. 12 depicts a plan view of an example of a fluid emission arresting device 101 further comprising a dispensing sheet 16 and a protective film 18 secured over the exterior surface 12 of the barrier layer 11 to a dispensing sheet 16 according to various embodiments described herein. In this and some embodiments, a protective film 18 may cover the barrier layer 11 and may be secured to a dispensing sheet 16 to prevent contamination, damage, and/or loss of a device 101. The protective film 18 may be temporarily secured to the dispensing sheet 16 and/or the device 101 allowing the protective film 18 to be secured over or to the barrier layer 11. When a user is ready to utilize the device 101, the protective film 18 may be peeled off or otherwise removed from covering the dispensing sheet 16 and the barrier layer 11 as shown in FIG. 12 thereby allowing the adhesive layer 14 (FIGS. 2 and 6) to be peeled off or otherwise removed from the dispensing sheet 16 as shown in FIG. 7.

FIG. 13 illustrates a plan view of an example of a plurality of fluid emission arresting devices 101 secured to a dispensing sheet 16 according to various alternative embodiments described herein. In this and some embodiments, a plurality such as twelve or more devices 101 may be secured to a dispensing sheet 16 to facilitate storage, transport, and/or transfer of the devices 101. In other embodiments, two, three, four, five, six, seven, eight, nine, ten, or eleven devices 101 may be secured to a dispensing sheet 16 to facilitate storage, transport, and/or transfer of the devices 101. In further embodiments, a protective film 18 (FIG. 12) may be temporarily secured to the dispensing sheet 16 and/or one or more of the devices 101 to prevent contamination, damage, and/or loss of a device 101.

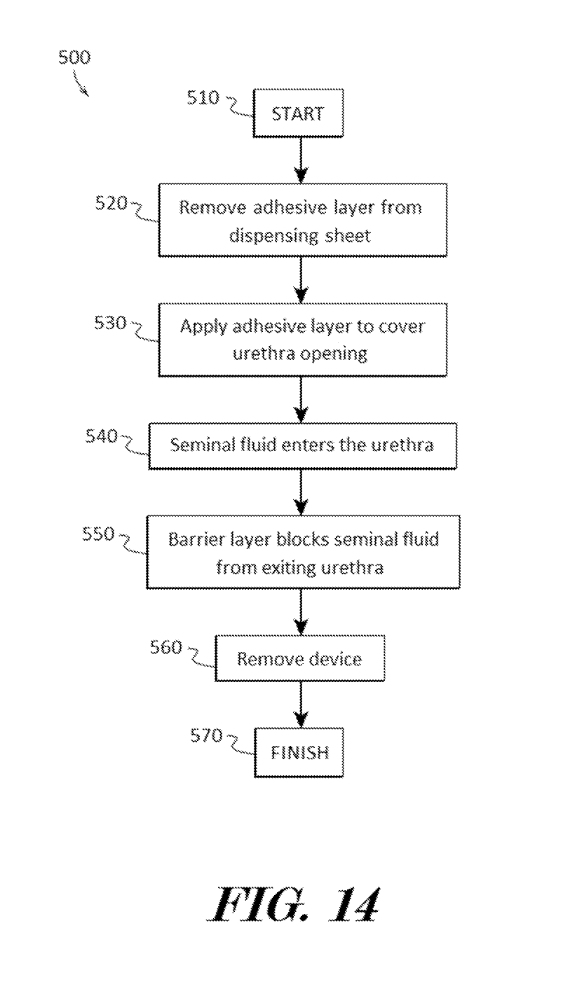

FIG. 14 shows a block diagram of an example of a method of arresting seminal fluid emission (“the method”) 500 with a fluid emission arresting system 100 (see FIG. 10) according to various embodiments described herein. In some embodiments, the method 500 may start 510 and the adhesive layer 14 (FIGS. 2, 7, and 9) may be removed from a dispensing sheet 16 (FIGS. 6, 7, 12, and 13) of a device 101 (FIGS. 1-13) in step 520. In further embodiments, a protective film 18 (FIG. 12) may also be removed from covering the exterior surface 12 (FIGS. 1-7, and 12) of the barrier layer 11 and/or the dispensing sheet 16. Next, the device 101 may be applied to the penis 200 (FIGS. 8-11) in step 530. In some embodiments, the device 101 may be applied to the penis 200 by contacting the adhesive layer 14 to the portions of the glans 201 (FIGS. 8-11) proximate to the urethra opening 203 (FIGS. 9-11), so that the adhesive layer 14 may secure the barrier layer 11 (FIGS. 1-11) over the urethra opening 203, thereby blocking seminal fluid 210 from exiting the urethra opening 203 (FIG. 10). In step 540, a bodily fluid such as seminal fluid 210 may enter the urethra 202. Seminal fluid 210 may then be blocked from exiting the urethra 202 as shown in FIG. 10 in step 550. In some embodiments, seminal fluid 210 may be prevented from exiting the urethra 202 through the urethra opening 203 by the adhesive layer 14 and the barrier layer 11 which are secured to the portions of the glans 201 which encircle the urethra opening 203. In this manner, seminal fluid 210 in the urethra 202, may be prevented from exiting the urethra opening 203 allowing the urethra 202 to function as a seminal fluid 210 reservoir. Seminal fluid 210 in the reservoir formed by the urethra 202 may therefore be prevented from exiting the urethra 202 until the device 101 is removed from covering the urethra opening 203 of the penis 200 in step 560. Once the device 101 is removed from covering the urethra 202, seminal fluid 210 and other bodily fluids may exit the urethra 202 and the method may finish 570.

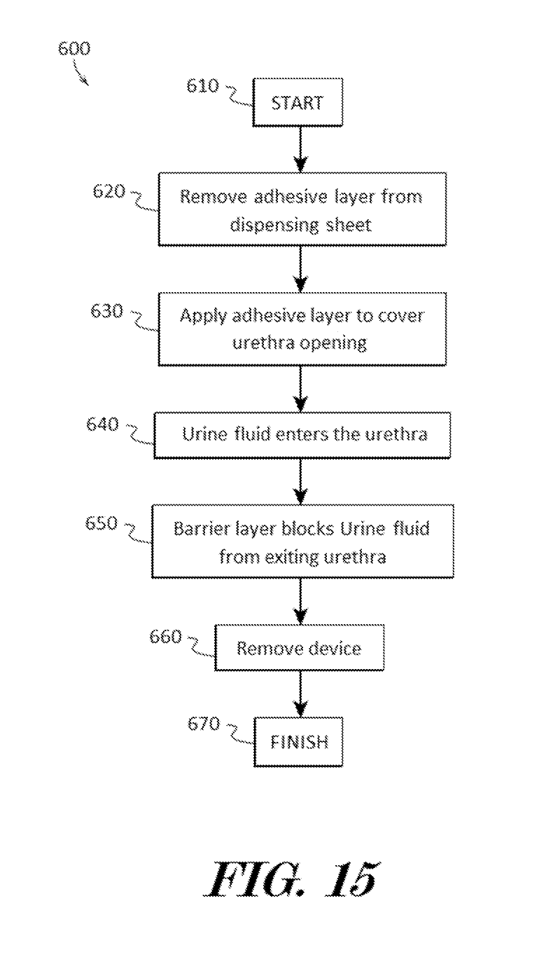

FIG. 15 depicts a block diagram of an example of a method of arresting urine fluid emission (“the method”) 600 with a fluid emission arresting system 100 (FIGS. 8-11) according to various embodiments described herein. In some embodiments, the method 600 may start 610 and the adhesive layer 14 (FIGS. 2, 7, and 9) may be removed from a dispensing sheet 16 (FIGS. 6, 7, 12, and 13) of a device 101 (FIGS. 1-13) in step 620. In further embodiments, a protective film 18 (FIG. 12) may also be removed from covering the exterior surface 12 (FIGS. 1-7, and 12) of the barrier layer 11 and/or the dispensing sheet 16. Next, the device 101 may be applied to the penis 200 (FIGS. 8-11) in step 630. In some embodiments, the device 101 may be applied to the penis 200 by contacting the adhesive layer 14 (FIGS. 2, 7, and 9) to the portions of the glans 201 (FIGS. 8-11) proximate to the urethra opening 203 (FIGS. 9-11), so that the adhesive layer 14 may secure the barrier layer 11 (FIGS. 1-11) over the urethra opening 203, thereby blocking urine fluid 220 from exiting the urethra opening 203 (FIG. 11). In step 640, a bodily fluid such as urine fluid 220 may enter the urethra 202. Urine fluid 220 may then be blocked from exiting the urethra 202 as shown in FIG. 11 in step 550. In some embodiments, urine fluid 220 may be prevented from exiting the urethra 202 through the urethra opening 203 by the adhesive layer 14 and the barrier layer 11 which are secured to the portions of the glans 201 which encircle the urethra opening 203. In this manner, urine fluid 220 in the urethra 202, may be prevented from exiting the urethra opening 203 allowing the urethra 202 to function as a urine fluid 220 reservoir. Urine fluid 220 in the reservoir formed by the urethra 202 may therefore be prevented from exiting the urethra 202 until the device 101 is removed from covering the urethra opening 203 of the penis 200 in step 660. Once the device 101 is removed from covering the urethra 202, urine fluid 220 and other bodily fluids may exit the urethra 202 and the method may finish 670.

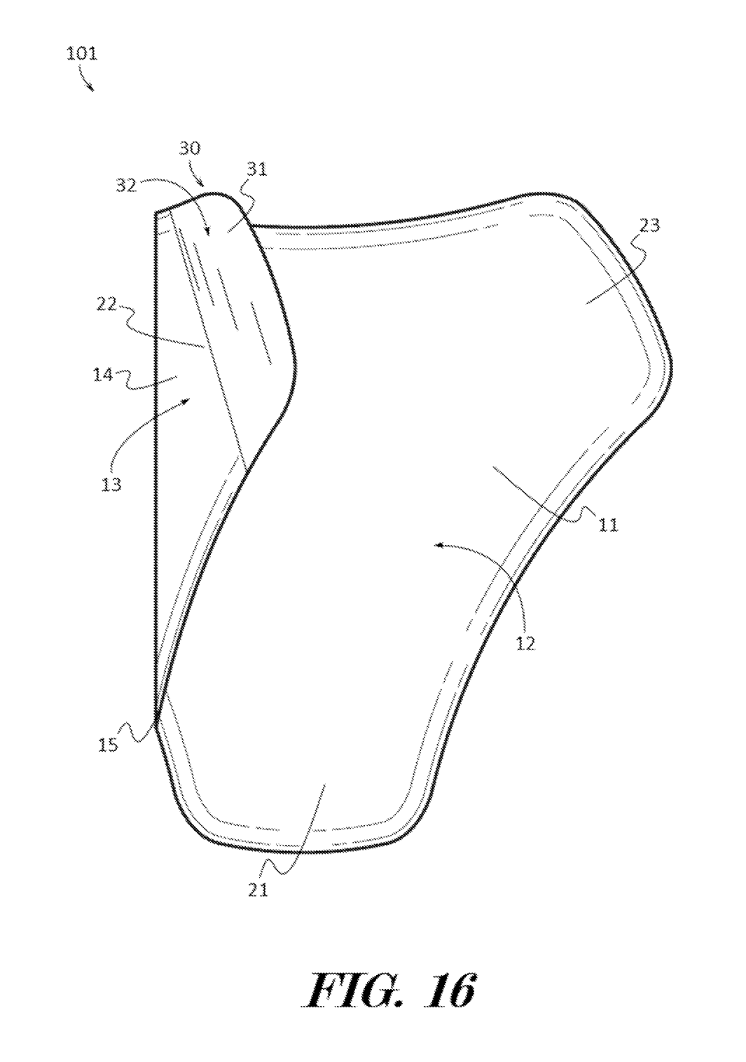

FIG. 16-FIG. 16 illustrates a top plan view of an alternative example of a fluid emission arresting device 101 according to various embodiments described herein. In this example, the device 101 comprises a barrier layer 11 with an exterior surface 12 and an interior surface 13. An adhesive layer 14 may be applied to the interior surface 13. The device 101 also includes a release tab 30 which may comprise a non-adhesive surface 31 which may be formed by or comprise a non-adhesive material 32. A release tab 30 may be configured to allow a portion of the interior surface 13 comprising a non-adhesive surface 31 to not adhere to objects such as skin, a dispensing sheet 16 (FIGS. 6, 7, 12, and 13), and the like. An optional release tab 30 may be used to facilitate the removal of the adhesive layer 14 from a dispensing sheet 16, skin, or other object, by allowing the user to grasp a portion of the exterior surface 11 and a portion of the interior surface 13 comprising the non-adhesive surface 31 while the adhesive layer is adhered to an object. The user may then pull the grasped release tab 30 to peel and remove the adhesive layer 14 and therefore the device 101 from the object to which it is adhered to. In some embodiments, a non-adhesive material 32 may comprise a paper gauze material. In other embodiments, a non-adhesive material 32 may comprise a thin sheet of paper, plastic, felt, fabric, or any other flexible film or sheet of material which does not comprise adhesive properties. In the embodiment shown in FIG. 16, a second lobe 22 may comprise a release tab 30. In other embodiments, a first lobe 21, second lobe 22, and/or a third lobe 23 may comprise a release tab 30. In still other embodiments, one or more release tabs 30 may be positioned anywhere along or proximate to the perimeter 15 of the device 101.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

What is claimed is:

1. A fluid emission arresting device, the device comprising:
a barrier layer made of a flexible material, wherein the barrier layer comprises a generally planar exterior surface and a generally planar interior surface;
an adhesive layer, wherein the adhesive layer is applied to the interior surface, wherein the adhesive layer is configured to attach to the glans of the penis to position the barrier layer over the urethra opening to block fluid from exiting the urethra opening.

2. The device of claim 1, wherein the barrier layer may prevent fluid from exiting the urethra so that the urethra may function as a fluid reservoir.

3. The device of claim 1, wherein the barrier layer is made from a film of material impermeable to bodily fluids.

4. The device of claim 1, wherein the barrier layer and adhesive layer are flexible and configured to conform the glans of the penis proximate to the urethra opening.

5. The device of claim 2, wherein the adhesive layer comprises a medical grade adhesive safe for contacting human skin.

6. The device of claim 1, wherein the adhesive layer comprises a medical grade adhesive that is impermeable to bodily fluids.

7. The device of claim 1, wherein the adhesive layer comprises a medical grade adhesive that is impermeable to pathogens.

8. The device of claim 2, wherein the adhesive layer is selected from one of an acrylic water-based medical grade adhesive and a pressure sensitive adhesive (PSA).

9. The device of claim 2, further comprising a dispensing sheet configured to be temporarily secured to the adhesive layer.

10. The device of claim 9, further comprising a protective film configured to be temporarily secured over the exterior surface of the barrier layer to the dispensing sheet.

11. A method of arresting fluid emission from a penis, the method comprising the steps of:
removing an adhesive layer from a dispensing sheet of a fluid emission arresting device comprising an adhesive layer, a barrier layer, and a dispensing sheet;
applying the adhesive layer to penis to cover urethra opening; and
blocking fluid from exiting the urethra with a barrier layer of a fluid emission arresting device affixed over the urethra opening by the adhesive layer.

12. The method of claim 1, wherein the barrier layer may prevent fluid from exiting the urethra so that the urethra may function as a fluid reservoir.

13. The method of claim 1, wherein the barrier layer is made from a film of material impermeable to bodily fluids.

14. The method of claim 1, wherein the barrier layer and adhesive layer are flexible and configured to conform the glans of the penis proximate to the urethra opening.

15. The method of claim 2, wherein the adhesive layer comprises a medical grade adhesive safe for contacting human skin.

16. The method of claim 1, wherein the adhesive layer comprises a medical grade adhesive that is impermeable to bodily fluids.

17. The method of claim 1, wherein the adhesive layer comprises a medical grade adhesive that is impermeable to pathogens.

18. The method of claim 2, wherein the adhesive layer comprises an acrylic water-based medical grade adhesive.

19. The method of claim 11, wherein the emission arresting device further comprises a protective film configured to be temporarily secured over the exterior surface of the barrier layer to the dispensing sheet.

20. The method of claim 19, wherein the method further comprises the step of removing the protective film from covering the exterior surface of the barrier layer.
